(19) United States
(12) Patent Application Publication
(10) Pub. No.: US 2008/0262619 A1
(43) Pub. Date: Oct. 23, 2008

(54) INTERSPINOUS PROCESS CUSHIONED SPACER

(76) Inventor: Charles D. Ray, Santa Barbara, CA (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/737,012

(22) Filed: Apr. 18, 2007

(51) Int. Cl. A61F 2/44

(52) U.S. Cl. 623/17.11

(57) ABSTRACT

An implantable dorsal spinous process spacer device including an implant body having a central portion, opposed superior legs, and opposed inferior legs. The central portion defines a lateral passage extending between, and exteriorly open to, left and right sides thereof. Optional cushions are affixed to opposite faces of the central portion, within receiving zones defined thereby. The implant body is attached to adjacent spinous processes. The implanted device spreads apart the posterior vertebral structures relieving a variety of disabling spine problems that have resulted from degenerative disc collapse. A narrow non-absorbable woven band, strap or plurality of suture filaments is passed around the two spinous processes, crossing through a lateral passage provided in the implant, stabilizing the spinal segment.

### FIELD OF THE INVENTION

Aspects of the present invention relate to an implantable spacer adapted for placement between spinous processes of the human spine, and more particularly, to a cushioned spacer with provision for its fixation and an optional, associated instrument for preparing the spinous process to accept the implant.

### BACKGROUND

Degenerative collapse of the human disc space is well known to be associated with disabling disc, facet joint and back pain. This is in part due to the buckling of annulus fibers of the disc, compromise or pinching of the exiting nerve space and an overriding of the posterior ancillary stabilizing facet joints attached in pairs to the vertebral bodies. Of the various forms of treatment, one of the more recently preferred methods involves placing a spacer between the posterior spinous processes in order to re-elevate the posterior structures, tightening the loosened fibers of the posterior disc annulus, spread open the exiting nerve foramen, and elevate the overriding facet joints. A variety of devices have been developed and successfully used as interspinous process spacers or spreaders that accomplish these desirable effects. Once implanted, it is imperative that the device not dislocate as a result of complex spine motions or the patient's lifting heavy objects. Therefore, some of the presently available devices developed for interspinal spacing are designed to prevent becoming displaced by being directly attached to the bony processes by clamps, wires, pins or screws. Unfortunately, such attachment means may lead to fracture or undesirable erosive changes in the spinous process bone.

An alternative approach is described in U.S. Pat. Nos. 6,761,720 and 6,946,000 in which an interspinous spacer system is described as including a multiperforated spacer to which is attached two belts. Each belt is assembled as a discrete, partial loop around an upper or lower spinous process, respectively, to an outer portion of the spacer for attachment and tightening. It is believed that this dual belt configuration lacks side shifting (translocation) resistance. The center mass of the relatively bulky spacer is provided in various sizes, the final size being determined by inserting a trial spacer among various sizes, without shaping the adjacent spinous process bones, A similar device was first reported by J Senegas in the European Spine Journal, October 2002, Supplement 2 pages 164-169 as “Mechanical supplementation by non-rigid fixation in degenerative intervertebral lumbar segments: the Wallis system,” and is currently undergoing clinical studies under the trade name Wallis® Mechanical Normalization System by the Abbott Spine Group, Austin Tex.

These and all other presently existing spinous spacer devices are constructed of metal or rigid polymers, and thus may damage the contacted bone over extended periods. Further, implantation of know spinous devices require the surgeon to perform delicate bone restructuring procedures in preparing/sizing the spinous processes for a close fit with the spacer device, especially where the spinous processes have worn against one another over time. This close fitting must be done carefully without perforating or fracturing the involved spinous processes. In the absence of appropriate tooling, this sizing procedure is difficult at best.

With the above Background in mind, improvements to, and advancement of, spinous process spacers and means for preparing the adjacent spinous processes, will be welcomed by spine surgeons and by appropriate patients alike.

### SUMMARY

Some aspects in accordance with principles of the present disclosure relate to an interspinous process spacer implant device adapted to be inserted between two adjacent spinous processes. The implant device comprises an implant body including a central portion, first and second superior legs, and first and second inferior legs. The central portion defines a left side, a right side, and a continuous, lateral passage extending through the central portion such that the lateral passage is open at both the left and right sides. The first and second superior legs extend upwardly from the central portion in an opposed, spaced fashion to define a superior zone for receiving a spinous process. The first and second inferior legs extend downwardly from the central portion in an opposed, spaced fashion to define an inferior zone for receiving a spinous process. With this configuration, the central portion is adapted to support opposing spinous processes disposed within the superior and inferior zones, respectively. In some embodiments, the lateral passage is sized to receive a separately-provided band. For example, the band (e.g., suture, strip, tape, etc.) is adapted for assembly through the lateral passage and about at least the first and second superior legs. In yet other embodiments, at least two segments of the band extend through the lateral passage, with other segments of the band extending about the superior legs and the inferior legs, defining a figure-8 construction, in securing the implant body to the adjacent spinous processes. In some embodiments, the implant device further includes a superior cushion disposed along the central portion between the first and second superior legs, and an inferior cushion positioned along the central portion between the inferior legs. The cushions are formed of a material that is softer than a material of the central portion (e.g., an elastomeric rubber), and reduce the point stresses placed upon the spinous process otherwise contacting the implant device.

Other embodiments in accordance with principles of the present disclosure relate to a kit for repairing a damaged vertebral column including adjacent vertebral segments each having a posterior spinous process. The kit includes first and second interspinous process spreader implant devices. Each of the implant devices includes a central portion, first and second superior legs, and first and second inferior legs. The first and second superior legs extend upwardly from the central portion in a spaced fashion to define a superior zone for receiving a spinous process. The first and second inferior legs extend downwardly from the central portion in a spaced fashion to define an inferior zone for receiving a spinous process. Finally, the central portion forms a continuous, lateral passage extending therethrough such that the lateral passage is open at both of a left side and a right side of the central portion. With this in mind, a lateral spacing between the superior legs of the first implant device is greater than a lateral spacing of the superior legs of the second implant device. With this configuration, upon evaluating a size of the spinous processes in question, the implant device providing a best fit thereto can be selected for subsequent implantation. In other embodiments, the kit further includes a bone shaping tool including a trough-like or U-shaped blade defining a lateral spacing commensurate with a lateral spacing between the superior legs of at least the first implant device.

Yet other aspects in accordance with principles of the present disclosure relates to a method of repairing a damaged vertebral column including adjacent superior and inferior vertebral segments each having a posterior spinous process. The method includes providing an interspinous process spacer implant device. The implant device includes an implant body having a central portion, first and second superior legs, and first and second inferior legs. The superior legs define a superior receiving zone, whereas the inferior legs define an inferior receiving zone. Further, the central portion defines a continuous, lateral passage extending through the central portion such that the lateral passage is open at both a left side and a right side thereof. With this in mind, the method further includes positioning the spinous process of the superior vertebral segment within the superior receiving zone, and positioning the spinous process of the inferior vertebral segment within the inferior receiving zone, A band is passed through the lateral passage and at least about the superior legs and the spinous process of the superior vertebral segment to secure the implant body to the superior vertebral segment. Upon final implant, the implant device establishes a near-normal spacing between the vertebral segments. In some embodiments, the method further includes extending a first segment of the band through the lateral passage; extending a second segment of the band around the superior legs and the spinous process of the superior vertebral segment; extending a third segment of the band through the lateral passage such that the first and third segments coexist within the lateral passage; and extending a fourth segment of the band about the inferior legs and the spinous process of the inferior vertebral segment. With this methodology, the band assumes a figure-8 shape.

Some non-limiting embodiments of the present disclosure provide an “H-shaped” spinous process spacer implant device in a plurality of sizes to accommodate a plurality of widths of the spinous processes, as encountered during spine surgery. The implant device in accordance with some embodiments also includes an interposed bioacceptable, elastomeric (rubbery) cushion formed within the valleys of the spacer at the central connecting area where there will be bone contact pressure. The elastomeric cushion bonded inside the vertical surfaces of the implant body can be an implantable grade silicone, polyurethane or polysulfone rubber or equivalent. The durometer rating of this elastomeric cushion should be moderately firm and withstand roughly 200-400 pounds psi compression repeatedly over several million cycles without fracture. Additionally, a lateral passage or slot is formed in the center of the spacer mass through which a resilient, bio-acceptable, non-absorbable band or multiple strands of suture may be passed. Such a band or bundle of sutures is brought above and below the adjacent spinous processes, then crosses through the spacer's mid portion slot, in an optional figure-eight style, stabilizing the implant and preventing its displacement. Where the spacer implant device is provided as part of a kit, an additional adjunct to the kit is one or more open “U” shaped sharp bone trims or tools that is used to prepare the spinous processes for a close fitting between the spinous process and the selected spacer implant device. During an implantation procedure, both the upper and lower facing, articulating spinous process margins are trimmed using the tool for proper fit inside the selected spacer and for optimal contact with the interposed elastomeric cushion. The plurality of widths of the novel bone trim cutters is matched with those of the spacers.

Regardless, over time, the bone of each wrapped spinous process is expected to show some accommodation or retreat of the bone where the relatively narrow, resilient band or suture bundle tightly passes. This remolding or accommodation will cause slight loosening of the band and an increase in flexion-extension angulation of the spine segment. This is a desirable effect of the present invention not found in other designs.

Other aspects of the present disclosure provide a spacer implant device having a low mass construction of a metal such as an implantable grade alloy of titanium or preferably an x-ray transparent, suitable polymer or composite such as carbon fiber reinforced Victrex® PEEK™ (polyetheretherketone) or epoxamide formulation. The torsional strength of the implant should be higher than that of the spinous process bone but still maintain substantial flexibility, greater than the elastic modulus of bone.

As a point of reference, if it is determined that the implant devices and methods described herein do not suffice for a particular patient, other means or methods may subsequently be employed, such as a bone fusion, or implantation of a prosthetic disc nucleus or a total artificial disc.

### BRIEF DESCRIPTION OF THE DRAWINGS

Embodiments of the invention are better understood with reference to the following drawings. The elements of the drawings are not necessarily to scale relative to each other. Like reference numerals designate corresponding similar parts.

FIG. 1 is a diagrammatic lateral view of lumbar vertebral segments Lumbar 4 (L4), Lumbar 5 (L5) and Sacral 1 (S1) having degenerated discs with narrowed annulus between the two lower disc spaces, L4-L5 and L5-S1. The simultaneous narrowing of the neural foramens and overlapping of facet joint pairs are shown. The interspinous ligaments for the segments immediately above and below the one to be operated are also indicated.

FIG. 2A is a front view of a portion of an interspinous process spacer implant device in accordance with principles of the present disclosure.

FIG. 2B is a side view of the implant device of FIG. 2A.

FIGS. 3A and 3B are a longitudinal cross-sectional view and a side view, respectively, of the implant device of FIG. 2A, illustrating partial assembly of a band to the implant body.

FIGS. 4A and 4B, and 5A and 5B, illustrate further assembly of the band to the implant body subsequent to the arrangement of FIGS. 3A and 3B.

FIG. 6A is a diagrammatic plane horizontal view of the distal end of an optional spinous process cutting and shaping tool that is matched with the inside contours of the spacer device selected.

FIG. 6B is an end view of the tool of FIG. 6A.

FIG. 7 is a diagrammatic lateral view of the vertebral column depicted in FIG. 1 now having the spacer implant device of FIG. 2A placed and strapped into position. At the operated level, the posterior annulus is now more normally separated, the associated foramen opened and the facet joint pairs have been returned to their normal position.

FIG. 8 is a diagrammatic direct posterior view of the final assembly showing the implant body and the stabilizing tape, band or sutures in position. The distracted, more normal positions of the disc space and facet joint pairs are shown. The close fitting of the implant device, prepared spinous process bone and cushions are also indicated. The interspinous ligaments of the spaces above and below the operated level remain essentially intact although they have been penetrated by the traversing strap or belt. The belt or strap is wound, figure-eight style and the final loose end bonded to itself by any of several known means.

For one skilled in the art, other implant and fixation designs may be substituted without changing the intent and performance of the invention.

### DETAILED DESCRIPTION

In the following Detailed Description, reference is made to the accompanying drawings that form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

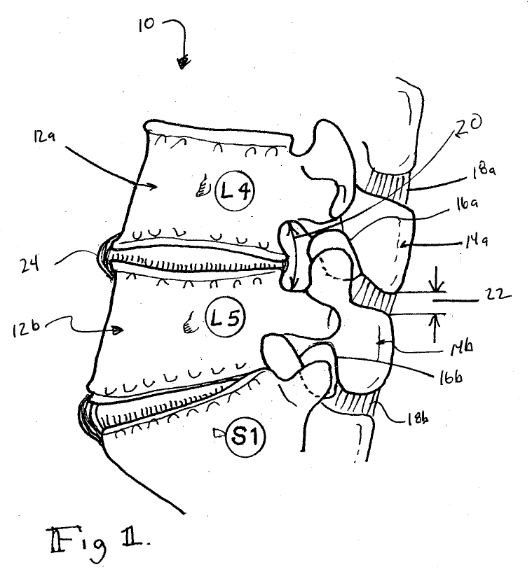

The present disclosure relates to an interspinous process spacer implant device useful in effectuating and maintaining an increased spacing between vertebral segments of a damaged vertebral segment of a damaged vertebral column. The implant device is described in detail below. As a point of initial reference, FIG. 1 illustrates a portion of a vertebral column 10 including adjacent vertebral segments 12a and 12b. While the implant device is useful in repairing damage evidenced at or between various adjacent segments of the vertebral column 10, FIG. 1 illustrates the adjacent segments 12a, 12b to be repaired as the L4 and L5 vertebrae, respectively, and further depicts sacral 1 (S1) segment. Each of the segments 12a, 12b of the vertebral column 10 has a posterior spinous process 14a, 14b, two facet joint pairs 16a, 16b and strong interspinous ligaments 18a, 18b. The narrowed and painful vertebral column segmental level in this illustration that is targeted for surgery lies between the vertebral segments 12a, 12b of L4 and L5. Here the neural foramens (passages for exit of the spinal nerve) 20 (referenced collectively in FIG. 1) are narrowed as well as is the interspinous ligament 22 and the annulus 24, particularly the posterior portion.

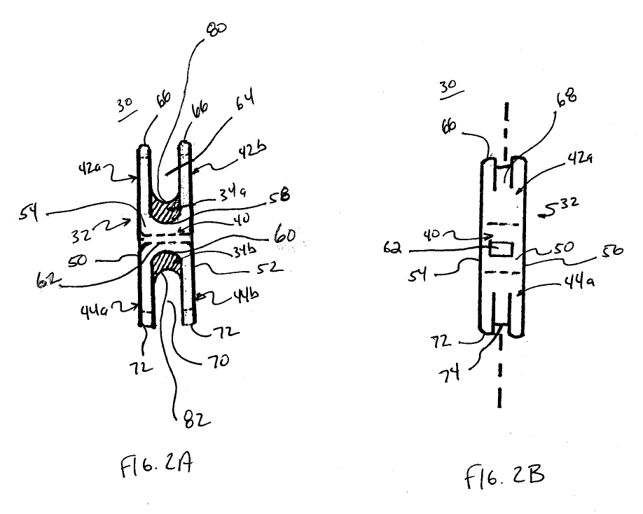

With the above anatomical description in mind, FIGS. 2A and 2B illustrate a portion of an implant device 30 in accordance with the present disclosure, and in particular an implant body 32 and optional cushions 34a, 34b. As described in greater detail below, the implant device 30 further includes one or more bands (not shown), such as a strap, tape, strip, suture, etc., useful in securing the implant body 32 to the spinous processes 14a, 14b (FIG. 1). In this regard, the cushions 34a, 34b, where provided, limit point stresses imparted by the implant body 32 upon the bony processes 14a, 14b.

The implant body 32 includes a central portion 40, superior legs 42a, 42b, and inferior legs 44a, 44b. In some embodiments, the components 40-44b are integrally formed of a biologically acceptable, highly rigid material such as an implant-grade alloy of titanium, a polymer reinforced with fibers (e.g., carbon fibers), etc. Regardless, the central portion 40 defines a left side 50, a right side 52, and anterior face 54, a posterior face 56, a superior face 58, and an inferior face 60. With these designations in mind, the central portion 40 further forms a continuous, lateral passage 62 extending between the left and right sides 50, 52. The lateral passage 62 is exteriorly open at both the left and right sides 50, 52 (it being understood that in the view of FIG. 2B, an opening to the lateral passage 62 at the left side 50 is depicted). The lateral passage 62 may have uniform dimension(s) in extension through the central portion 40, or variations thereof may be provided. Regardless, the lateral passage 62 is sized in accordance with a size of the band (not shown) such that in some embodiments, two or more segments of the band can co-exist in extension through the lateral passage 62. Similarly, while the lateral passage 62 preferably extends in a generally perpendicular fashion relative to a plane defined by either of the left or right sides 50, 52, in other embodiments, an angular off-set can be established.

In some embodiments, the lateral passage 62 is centered between the superior and inferior faces 58, 60 and/or centered between the exposed surfaces of the cushions 34a, 34b. In other embodiments, however, the lateral passage 62 can be positioned closer to one or the other of the superior or inferior faces 58 or 60. Finally, and as best shown in FIG. 2B, the lateral passage 62 is centered between the anterior and posterior faces 54, 56. In other embodiments, the lateral passage 62 can be positioned in closer proximity to one or the other of the anterior or posterior faces 54, 56.

Apart from the lateral passage 62 described above, the central portion 40 is preferably characterized by the absence of other slots or apertures extending through a thickness or width thereof. More particularly, the central portion 40 does not, in some embodiments, include any continuous bores or passages extending completely between, and open at both of, the anterior and posterior faces 54, 56. With this one construction, and in combination with the superior and inferior legs 42a, 42b, 44a, 44b as described below, the implant body 32 has an H-shape when viewed posteriorly or anteriorly. In other embodiments, however, one or more partial or complete bores or passages through the central portion 40, in addition to the lateral passage 62, can be provided.

The superior legs 42a, 42b extend upwardly from the central portion 40 in a spaced fashion. In some embodiments, the first superior leg 42a is formed as a continuation of the left side 50 of the central portion 40, whereas the second superior leg 42b is a continuation of the right side 52. Regardless, a transverse spacing is established between the superior legs 42a, 42b, defining a superior receiving zone 64 sized to receive a portion of a spinous process. Each of the superior legs 42a, 42b terminates at a tip 66 opposite the superior face 58 of the central portion 50. In some embodiments, the superior legs 42a, 42b have a substantially identical length in extension to the corresponding tip 66; alternatively, the superior legs 42a, 42b can have different dimensions. Regardless, and as best shown in FIG. 2B, in some embodiments, each of the superior legs 42a, 42b forms a tapered groove 68 extending from and including the corresponding tip 66 (it being understood that in the view of FIG. 2B, the groove 68 for the first superior leg 42a is shown). As described in greater detail below, the groove 68 is sized to receive one or more segments of the band (not shown) otherwise used in securing the implant body 32 to the vertebral column 10 (FIG. 1). Alternatively, however, the groove 68 can be eliminated.

The inferior legs 44a, 44b are, in some embodiments, identical to the superior legs 42a, 42b described above, and extend downwardly from the central portion 40. Thus, the inferior legs 44a, 44b are transversely spaced from one another, combining to define an inferior receiving zone 70 sized to receive a spinous process. The transverse spacing defined between the inferior legs 44a, 44b (i.e., width of the inferior receiving zone 70) is preferably substantially identical to that defined between the superior legs 42a, 42b (e.g., within 5% of an identical size); alternatively, the inferior receiving zone 70 can be substantially (e.g., greater than 10%) wider or narrower than the superior receiving zone 64. As with the superior legs 42a, 42b, the inferior legs 44a, 44b each terminate at a tip 72 opposite the inferior surface 60, and each optionally forms a tapered groove 74 (shown for the first inferior leg 44a in FIG. 2B.

With the above construction of the implant body 32 in mind, the optional cushions 34a, 34b are secured (e.g., bonded) to the implant body 32 as shown. More particularly, the first cushion 34a is secured to the superior face 58 of the central portion 40, thus defining an abutment surface 80 of the superior zone 64. Conversely, the second cushion 34b is affixed to the inferior face 60 of the central portion 40, and thus defines an abutment surface 82 of the inferior zone 70.

The cushions 34a, 34b effectively serve as shock absorbers for the contacted spinous processes following implant. The cushions 34a, 34b are formed of a softer material as compared to that of the implant body 32. In some embodiments, the cushions 34a, 34b are formed of elastomeric rubber material, for example, an implant grade silicone, polyurethane, or polysulfone rubber or other material exhibiting a durometer rating that is moderately firm and able to withstand roughly 200-400 psi compression repeatedly over several million cycles without fracture. In other embodiments, however, one or both of the cushions 34a and/or 34b can be eliminated. Further, while the cushions 34a, 34b are illustrated in FIG. 2A as forming the corresponding abutment surface 80, 82 as having a continuous curvature, in other embodiments, a linear or complex curved surface can be formed. Preferably, however, sufficient cushion material provided to support edges of a spinous process located within the corresponding receiving zone 64, 70.

Implantation of the device 30 to the vertebral column 10 (FIG. 1) is described in greater detail below. One technique for securing the implant body 32 using a band 90 is provided in FIGS. 3A-5B. In general terms, the band 90 is wrapped about and through the implant body 32 via the lateral passage 62. To better illustrate one desired assembly technique, the band 90 is depicted in FIGS. 3A-5B as being loosely associated/connected with the implant body 32, it being understood that in actual practice, the band 90 can be more tightly associated with the implant body 32 (as well as to the vertebral column 10 (FIG. 1) being repaired). With this in mind, FIG. 3A illustrates the band 90 (e.g., a suture, tape, belt, etc.) initially secured to the implant body 32, such that a first segment 92 extends through the lateral passage 62 (e.g., extending from the right side 52 to the left side 50), and a second segment 94 extends around or about the superior legs 42a, 42b. As shown in FIG. 3B, the band 90 can be located within the groove 68 of the first superior leg 42a.

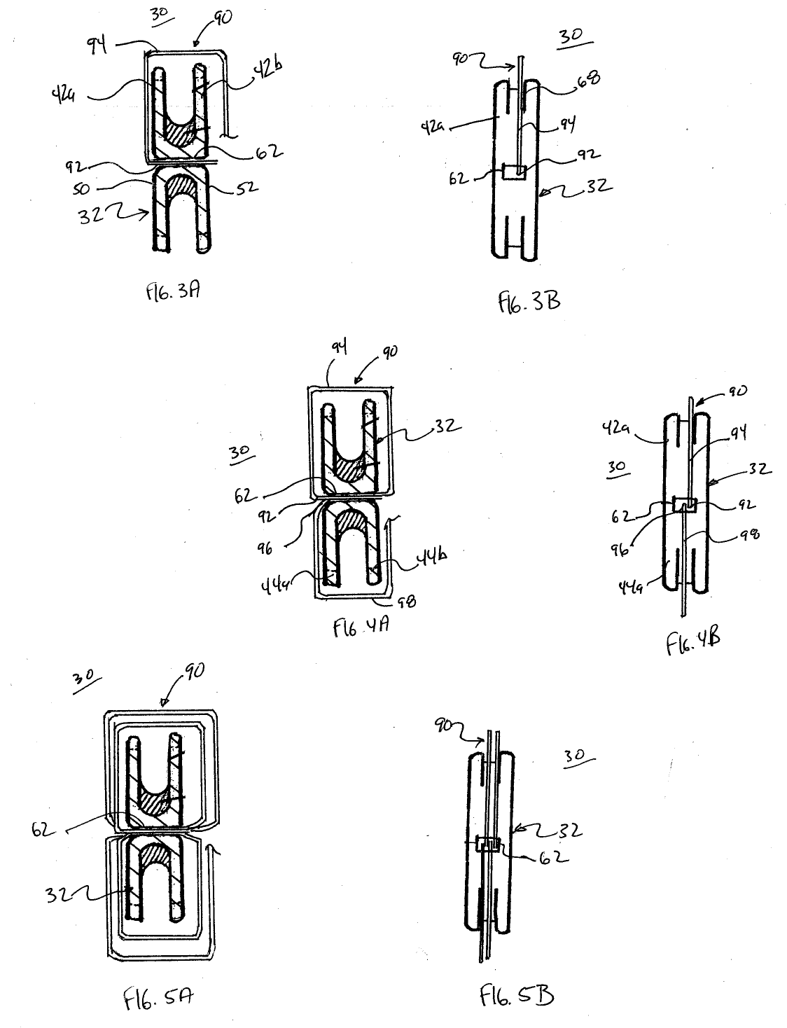

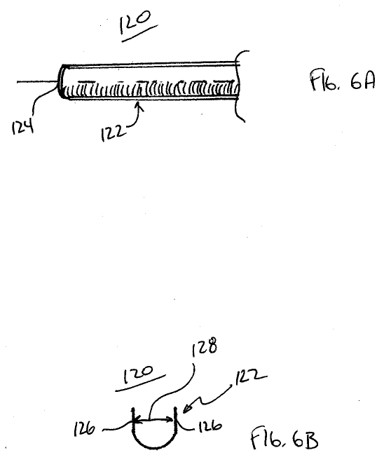

In FIGS. 4A and 4B, the band 90 is subsequently articulated to define a third segment 96 extending through the lateral passage 62, and a fourth segment 98 extending around or about the inferior legs 44a, 44b. As shown in FIG. 4B, then, the lateral passage 62 is appropriately sized such that the first and third segments 92, 96 simultaneously extend through, or coexist within, the lateral passage 62. Where desired, and as reflected in FIGS. 5A and 5B, the band 90 can be further wrapped about the superior legs 42a, 42b and the inferior legs 44a, 44b in a repeating manner, each time passing through the lateral passage 62, to establish a figure-8 type construction. While the implant device 30 has been described as using a single one of the bands 90 in securing the implant body 32, in other embodiments, two or more bands can be provided (e.g., a first band can be wrapped one or more times about the superior legs 42a, 42b, passing through the lateral passage 62, whereas a second band can be wrapped or wound about the inferior legs 44a, 44b, again passing through the lateral passage 62). The band 90 can be secured to the implant body 32 in various manners, such as by tying the opposed ends of the band 90 to one another, wrapping the band 90 on to itself so as to “lock” free ends thereof relative to the implant body 32. With these and other similar techniques, a rigorous securement of the band 90 to the implant body 32 is of less concern due to, in some embodiments, a frictional interface between the band 90 and the implant body 32 and/or bone or tissue in-growth that further holds the band 90 to the implant body 32. In other embodiments, however, a separate fastener, such as a surgical clip, can be provided.

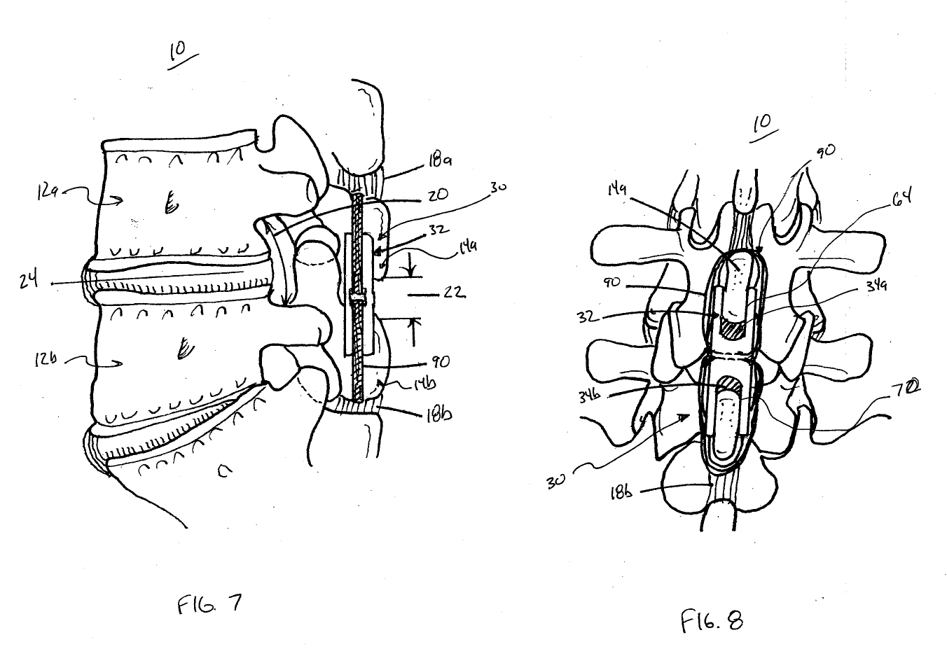

To assist in achieving a best fit between the implant device and the spinous processes in question, an optional shaping tool 120 can be provided as shown in FIGS. 6A and 6B. More particularly, a distal, blade end 122 of the tool 120 is illustrated, forming a sharpened distal tip 124 and having a trough-like or U-shape. With this construction, the blade 122 provides opposing walls 126 defining a transverse spacing 128 (best shown in FIG. 6B). The transverse spacing 126, in turn, corresponds with a transverse spacing provided by the superior and inferior receiving zones 64, 70 (FIG. 2A). During use, then, the tool 120 can be used to prepare the spinous processes, shaving excess bony material, and shaping the spinous processes to closely fit within the corresponding receiving zone 64 or 70. Where provided, the tool 120 can be highly useful is shaping the edges of spinous processes that have been in contact with one another over time, and are thus have a worn, flattened or splayed surface.

In some embodiments, the implant device 30 (FIG. 2A) is provided as part of a kit for repairing a damaged vertebral column. More particularly, two or more of the implant devices 30 are provided, with the implant devices being essentially identical except having different transverse spacing characteristics. For example, the transverse spacing provided by the superior and inferior receiving zones 64, 70 of the first implant device can be greater than or less than the corresponding transverse spacing provided by the second implant device. Further, the kit can include two or more of the tools 120 (FIGS. 6A and 6B) each one of which has a blade end 122 sized in accordance with a corresponding one of the implant devices. Further, the kit can include instructions for use.

In other embodiments, the implant device 30 can have a self-spacing or expanding feature such that a single implant device 30 can be used with a variety of differently-sized patients. For example, and with specific reference to FIG. 2A, the central portion 40 can be formed or defined by two components that are slidably (e.g., telescopically) connected to another. More particularly, a first half of the central portion can define the left side 50 and carry or include the first superior leg 42a and the first inferior leg 44a. The second half defines the right side 52 and carries or includes the second superior leg 42b and the second inferior leg 44b. The halves are slidably connected to one another such that a lateral spacing or size of the receiving zones 64, 70 can be altered with movement of the central portion segments relative to one another. With this configuration, prior to implant, the device is arranged in an expanded state, having a near maximum lateral spacing, with the left side 50 being pulled or slid away from the right side 52. The enlarged receiving zones 64, 70 are then situated about a corresponding spinous process. The band 90 is then wrapped about the implant body as described above. Tightening of the band 90 causes the implant device to retract, with the left and right sides 50, 52 being forced toward one another. Retraction continues until the legs 42a, 42b and 44a, 44b contact and engage the spinous process disposed therebetween. Thus, this alternative configuration provides for a self-spacing feature, and can readily be deployed at the implant site.

Regardless of an exact construction, FIG. 7 is an additional lateral plan view of the vertebral column 10 of FIG. 1 shown after implantation of the implant device 30. The spinous process 14a of the superior segment 12a is disposed within the superior zone 64, in contact with and supported by the cushion 34a. Similarly, the spinous process 14b of the inferior segment 12b is disposed within the inferior zone 70, in contact with and supported by the cushion 34b. The neural foramen 20 and annulus 24 have been substantially elevated or opened by the implant device 30 which has been stabilized using the strap or band 90 placed around the upper and lower margins of the adjacent spinous processes 14a, 14b, passing through but not disrupting the interspinous ligaments 18a, 18b at those levels. The spacer implant device 30 creates and maintains a substantial distance between the involved spinous processes 14a, 14b and elevation of the interspinous ligament 22, improving the clinical status of the patient.

FIG. 8 is a posterior plan view of the vertebral column 10, as in FIG. 7, showing the components of the spacer implant device 30 and the involved anatomy. The spinous processes 14a, 14b have been prepared, while the interspinous process ligaments 18a, 18b of adjacent levels remain essentially intact. The implant device 30 with its interposed cushions 34a, 34b is shown, fixed in position and stabilized by the figure-eight style wrapping (in one embodiment) of the strap or belt 90 as described above. Other aspects of the anatomy of the vertebral column 10 are also depicted.

### EXAMPLE OF USE

An otherwise healthy, suitable patient having disc collapse with associated discogenic pain or other vertebral column 10 damage at adjacent vertebral segments 12a, 12b is selected. With reference to FIGS. 7 and 8, the interspinous ligament 18a, 18b at the surgical level alone is surgically removed and the opposing spinous processes 14a, 14b are suitably prepared using, for example the tool 120 (FIGS. 6A and 6B) whose width and contour are matched to that of the chosen implant device 30. The implant device 30 is positioned, with the interposed elastomeric cushions 34a, 34b firmly against the end surfaces of the prepared spinous processes 14a, 14b, and the implant device 30 is anchored in place using multiple passes of a narrow non-absorbable band or tape 90, such as 2 mm or 3 mm wide, or a bundle of individually placed strong #1 sutures sewn through the remaining interspinous ligaments above and below the operated space. This non-slip banding 90 crosses the approximate midline, through the lateral passage 62 of the implant device 30, figure-eight style in some embodiments, and is tied or fixed at its loose end to prevent unraveling. By crossing the approximate midline in this way, torsional displacements of the spinous processes 14a, 14b or distracted facet joints 16a, 16b are prevented. The posterior aspect of the annulus 24 then has become distracted and its fibers tightened, restoring considerable stability to the disc itself and thus the vertebral segment 12a, 12b. The disc and facet joints and the neural foramens are now returned to more normal positions.

With anticipated accommodation of the bone of the spinous processes to the tight band, tape or sutures, the tight configuration will slacken slightly allowing more freedom of spinal column flexion-extension, a desirable trait.

### ADVANTAGES

The invention has the ability to restore the height of the posterior annulus of the disc and open the facet joints and exiting nerve foramens while stabilizing the construct with a band of tape or sutures placed in, for example, a figure-eight fashion around the spinous processes and across through a central slot in the implant to the other side. This configuration restores and establishes considerable torsional strength to the degenerated spinal segment and over time will slowly permit some desirable increase of flexion-extension mobility to the segment. The implant uniquely provides, in some embodiments, interposed elastomeric rubber shock absorbers facing the prepared ends of the spinous processes.

The implantation method is minimally invasive, performed through a small posterior incision, perhaps under local anesthesia and is extremely simple for spine surgeons. The implantation is facilitated by using the optional bone-shaping and trimming tool that is under control by the surgeon at all times. The device and method can be used with or without temporary adjunctive external bracing as required by the particular patient. The surgeon may apply the tape, band or sutures as tightly or loosely as desired.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

The application of the spinous process spacer implant device is devoid of any undesirable side effect such as rejection of the implant materials, an inadvertent subsequent promotion of a segmental fusion or uncomfortable overlying muscle abrasion that can occur by protruding parts of implants. The segmental stabilizing and pain relieving effects are immediate and continuous. The implant and the optional elastomeric bumpers can be constructed and attached together in a variety of ways. Preferred means to prepare for and install the implant are disclosed here although persons skilled in the mechanical arts can adapt the concept to a variety of means to cause desirable alternative means.

No other inter spinous spreading device or method serving this application or in combination with the interposed elastomeric bumper and means of strapping fixation between the spinous processes under surgeon control is known to exist at this time.

What is claimed is:

1. An interspinous process spacer implant device adapted to be inserted between two adjacent spinous processes, the implant device comprising:
an implant body including:
a central portion defining a left side, a right side, an anterior face, and a posterior face;
opposing first and second superior legs extending upwardly from the central portion in a spaced fashion to define a superior zone for receiving a spinous process;
opposing first and second inferior legs extending downwardly from the central portion in a spaced fashion to define an inferior zone for receiving a spinous process;
wherein the central portion is adapted to support spinous processes disposed within the superior and inferior zones, respectively, and forms a continuous, lateral passage extending through the central portion such that the lateral passage is open at both the left and right sides.

2. The implant device of claim 1, wherein the lateral passage is sized to receive a band.

3. The implant device of claim 2, further comprising:
a band adapted for assembly through the lateral passage and about at least the first and second superior legs.

4. The implant device of claim 3, wherein the band is further adapted for assembly about the first and second inferior legs.

5. The implant device of claim 4, wherein the band is adapted for assembly about the inferior legs and the superior legs, and through the lateral passage in a figure-8 manner.

6. The implant device of claim 3, wherein the lateral passage and the band are sized such that at least two segments of the band can simultaneously be disposed within the lateral passage.

7. The implant device of claim 3, wherein the first superior leg forms a continuation of the left side of the central portion and the second superior leg forms a continuation of the right side of the central portion, each of the superior legs terminating at a tip opposite the central portion, and further wherein each of the superior legs forms a groove at the tip for receiving the band.

8. The implant device of claim 3, wherein the band is a suture.

9. The implant device of claim 1, wherein the central portion further defines a superior face between the first and second superior legs, the implant device further comprising:
a superior cushion affixed to the superior face, the superior cushion defining an abutment surface adapted to contact a spinous process disposed within the superior zone.

10. The implant device of claim 9, wherein the superior cushion is formed of a material that is softer than a material of the implant body.

11. The implant device of claim 10, wherein the superior cushion is an elastomeric rubber.

12. The implant device of claim 9, wherein the central portion further defines an inferior face between the first and second inferior legs, the implant device further comprising:
an inferior cushion affixed to the inferior face, the inferior cushion defining an abutment surface adapted to contact a spinous process disposed within the inferior zone.

13. The implant device of claim 1, wherein the first superior leg and the first inferior leg form a continuation of the left side of the central portion, the second superior leg and the second inferior leg form a continuation of the right side of the central portion such that the legs and the central portion combine to define an H-shape.

14. The implant device of claim 13, wherein the central portion is characterized by the absence of an opening extending continuously through and between the anterior and posterior faces.

15. A kit for repairing a damaged vertebral column including adjacent vertebral segments each having a posterior spinous process, the kit comprising:
first and second interspinous process spacer implant devices, each comprising:
an implant body including:
a central portion defining a left side, a right side, an anterior face, and a posterior face;
opposing first and second superior legs extending upwardly from the central portion in a spaced fashion to define a superior zone for receiving a spinous process;
opposing first and second inferior legs extending downwardly from the central portion in a spaced fashion to define an inferior zone for receiving a spinous process;
wherein the central portion is adapted to support spinous processes disposed within the superior and inferior zones, respectively, and forms a continuous, lateral passage extending through the central portion such that the lateral passage is open at both the left and right sides;
wherein a lateral spacing between the superior legs of the first implant device is greater than a lateral spacing between the superior legs of the second implant device.

16. The kit of claim 15, further comprising:
a band adapted for assembly to either of the first and second implant devices, including extension through the corresponding lateral passage.

17. The kit of claim 15, further comprising:
a bone shaping tool including a trough-like blade end.

18. The kit of claim 17, wherein the trough-like blade end defines a lateral spacing commensurate with a lateral spacing between the superior legs of the first implant device.

19. A method of repairing a damaged vertebral column including adjacent superior and inferior vertebral segments each having a posterior spinous process, the method comprising:
providing an interspinous process spacer implant device comprising an implant body including a central portion, opposing superior legs, and opposing inferior legs, wherein the superior legs extend upwardly from the central portion in a spaced fashion to define a superior zone, and the inferior legs extend downwardly from the central portion in spaced fashion to define an inferior zone;
positioning the spinous process of the superior vertebral segment within the superior zone;
positioning the spinous process of the inferior vertebral segment within the inferior zone; and
passing a band through the lateral passage and about the superior legs to secure the implant device to the superior vertebral segment;
wherein upon final implant the implant device establishes and maintains a spacing between the vertebral segments.

20. The method of claim 19, wherein passing the band includes:
extending a first segment of the band through the lateral passage;
extending a second segment of the band around the superior legs and the spinous process of the superior vertebral segment;
extending a third segment of the band through the lateral passage such that the first and third segments co-exist within the lateral passage; and
extending a fourth segment of the band about the inferior legs and the spinous process of the inferior vertebral segment.
